(19) United States
(12) Patent Application Publication
(10) Pub. No.: US 2001/0034896 A1
(43) Pub. Date: Nov. 1, 2001

(54) Swimming goggles with step-less adjustment

(76) Inventor: Herman Chiang, Chung-Ho City (TW)

Correspondence Address: Keith Kline, PRO-TECHTOR INTERNATIONAL SERVICES, 20775 Norada Court, Saratoga, CA, 95070-3018, US

(21) Appl. No.: 09/798,841

(22) Filed: Mar. 1, 2001

Related U.S. Application Data
(63) Continuation-in-part of application No. 09/541,233, filed on Apr. 3, 2000.

(51) Int. Cl. A61F 9/02

(52) U.S. Cl. 2/428

(57) ABSTRACT

A type of swimming goggles, comprises: two lens frame units, each lens frame unit accommodates a lens, and, at its two opposite ends are a first joining unit and a second joining unit, wherein the first joining which is located closely to the inner of each lens frame has a through hole, and the second joining units which located on the outside of each lens frame has a engaging hole with a clasp opening thereon; which the diameter was less than the through holes and the a engaging hole, to pull and position in sequential order along the through holes and the clasp opening of the first and second joining units of said two lens frame units, maintaining an appropriate span between the two lens frame units; and a headband device that is joined to the two joining units on the outside of the two lens frame units, so as to enabling step-less adjustment to suit the nose bridge configuration of different users who can feel comfortable, and can be more convenient and do not effect the span after adjusting the span.

### CROSS REFERENCE OF RELATED APPLICATION

This application is a continuation in part application of U.S. patent application Ser. No. 09/541,233 filed on Apr. 3, 2000.

### BACKGROUND OF THE INVENTION

1. Field of the Invention

The structural feature of swimming goggles in U.S. patent application Ser. No. 09/541,233 is as shown in FIG. 1, To provide at two ends of the longer axis of the lens frame 500,510 of the swimming goggle has a clasp opening 501,511, and a clasp piece 502,512 is formed the up and down rim of the lens frame 500,510 respectively. Each of the clasp piece 502,512 and the clasp opening 501,511 defines an engaging hole for two string 60,61 position therein, thereby forming a nose bridge of swimming goggle by the two string pass through the lens frame 500,510. There two way to adjust the distance of the string 60,61 to match a nose bridge before wearing, one is to separate each of the string 60,61 from the clasp opening 501,511 then the adjustment and moving get an appropriate distance to match a nose bridge from each the free ends of the string 60,61, also engage the string 60,61 into the clasp piece 501,511 respectively. The other way is just separate the string 60,61 from the clasp piece 511 and adjust an appropriate distance to match a nose bridge from near the clasp piece 501 at the string 60,61, also engage the string 60,61 into the clasp piece 511 respectively. Therefore the string 60,61 can be adjusted with different span to match user's nose bridge, and user will get more comfortable of wear.

However, one of the drawbacks of the aforementioned swimming goggle resides in that the diameter of the engaging hole with the clasp piece 501,511 is 2 mm, and the diameter of the string 60,61 is also 2 mm, hence the diameter of the string 60,61 is larger than the clasp piece 501,511 of the engaging hole. The string should be pressed with big effort into the clasp piece 501,511.in force, in order to securely firm the string 60,61 in the engaging hole. Accordingly, It will leads to the span change which can not comply with a nose bridge of user, due to the small space in near the clasp piece 501 inconvenience for operation, after the adjustment be well and engaging the string 60,61 into the clasp piece 501.

Another drawback of the aforementioned swimming goggle resides in that the string 60,61 are moved so hard in the engaging hole, leads to inconvenience the adjustment a distance, in view of the string 60,61 are clipped by the clasp piece 501 of the engaging hole, when someone adapt to separate the string 60,61 from the clasp piece 511.

### BRIEF DESCRIPTION OF THE INVENTION

The primary objective of the subject invention of swimming goggles with step-less adjustment is to improve the form of the clasp piece which closely the inner of the lens frame unit, and so as to get more smooth during each string pass through said clasp piece, Thereby it can be more convenience and do not effect the span after the adjustment of the span.

### CHARACTERISTICS OF THE INVENTION

The subject invention of swimming goggles with step-less adjustment comprise a pair of lens frame having a first joining units and a second joining units at two ends of the longer axis of each lens frame, wherein the first joining units which is close to the inner of each lens frames has a through hole, and the diameter of the through hole correspond to the diameter of a engaging hole of the second joining units. The engaging hole having a clasp opening within, is located to close to outside of each lens frames. Also the diameter of a string of swimming goggle is less than the through hole and the engaging hole. Therefore, both the free end of each string should smoothly pass through the through hole of the first joining units respectively, then clipping each free end into the clasp opening of the engaging hole respectively. When user would like to adjust the distance of nose bridge, he/she just separate the free end of each string from a clasp piece and then, adjust and move the strip to get an appropriate distance to match his/her nose bridge, finally engage each string into the clasp opening of the engaging hole respectively.

### BRIEF DESCRIPTION OF DRAWINGS

FIG. 1 is a perspective view of the U.S. patent application Ser. No. 09/541,233.

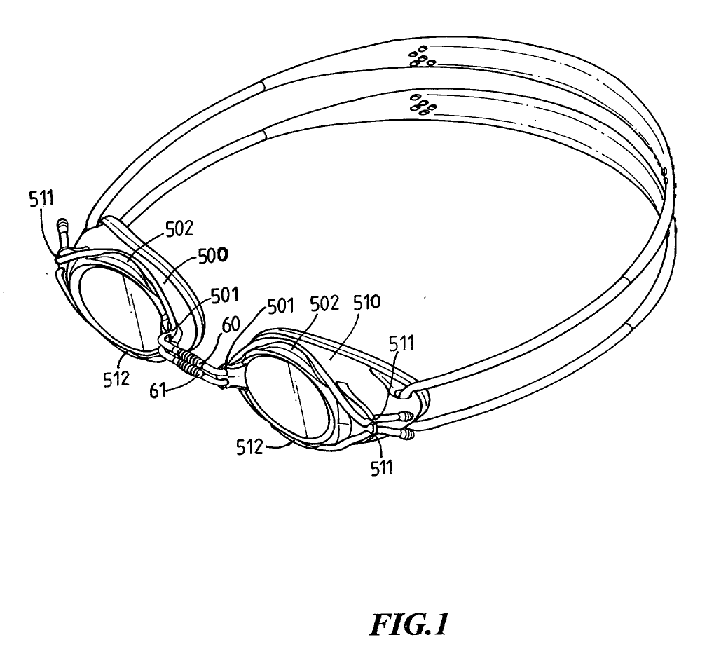

FIG. 2 is a perspective view of the disassembled parts of the invention of swimming goggles with step-less adjustment.

FIG. 3 is a perspective view of the assembled of the invention of swimming goggles with step-less adjustment.

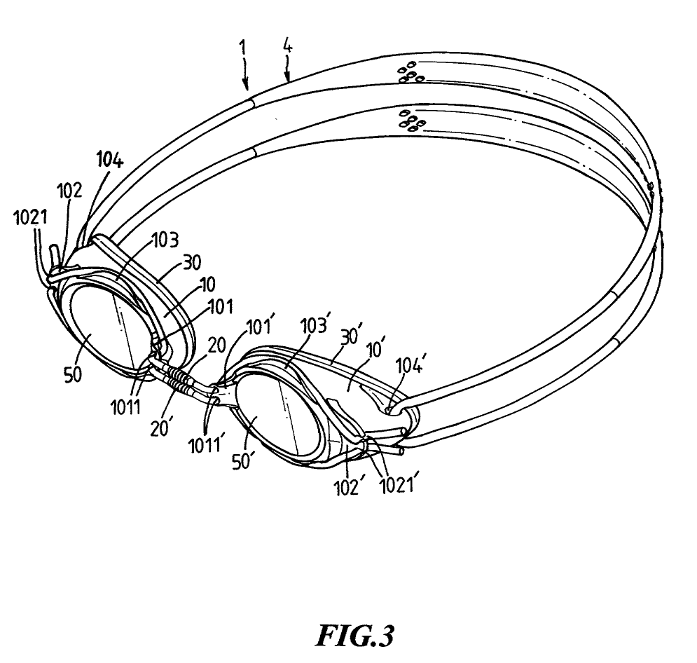

FIG. 4 is a front view of the invention of swimming goggles with step-less adjustment.

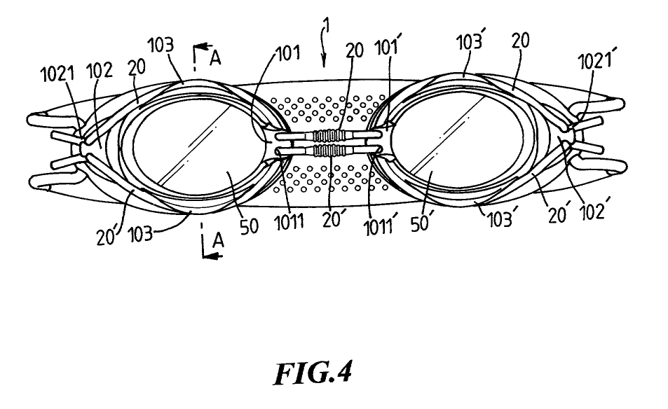

FIG. 5 is a perspective view of the difference span of both string which show the length of each string to suit the user's nose bridge contour.

### BRIEF DESCRIPTION OF NUMERALS

| | | |
|---|---|---|
| 1 | swimming goggles | 10, 10′ lens frame unit |
| 20, 20′ | string | |
| 3 | protective pad | 30, 30′ face contact part |
| 4 | headband device | 50, 50′ lens |

| | |
|---|---|
| 101, 101′ | first joining unit |
| 102, 102′ | second joining unit |
| 1011, 1011′ | through hole |
| 1021, 1021′ | clasp opening |
| 103, 103′ | clasp piece |
| 104, 104′ | headband through hole |

### DETAILED DESCRIPTION OF PREFERRED EMBODIMENT

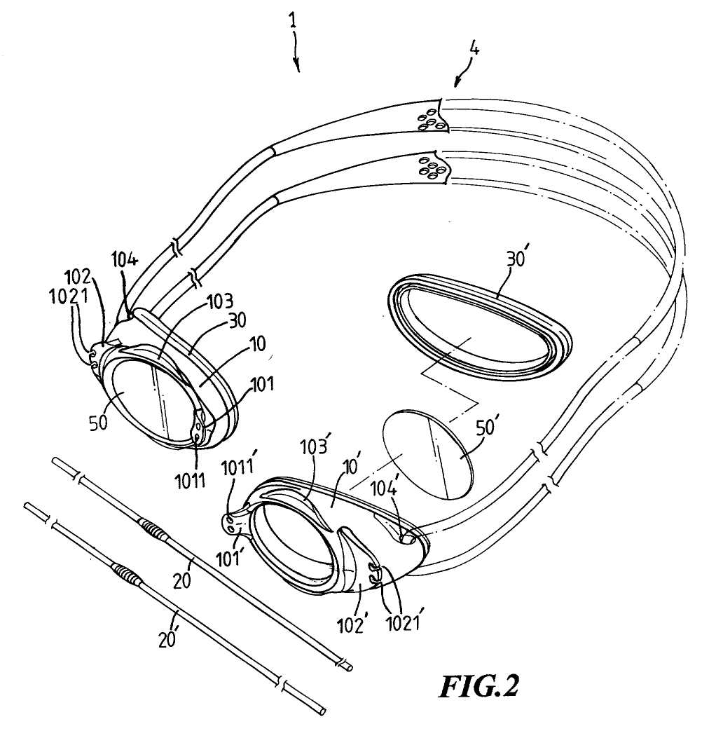

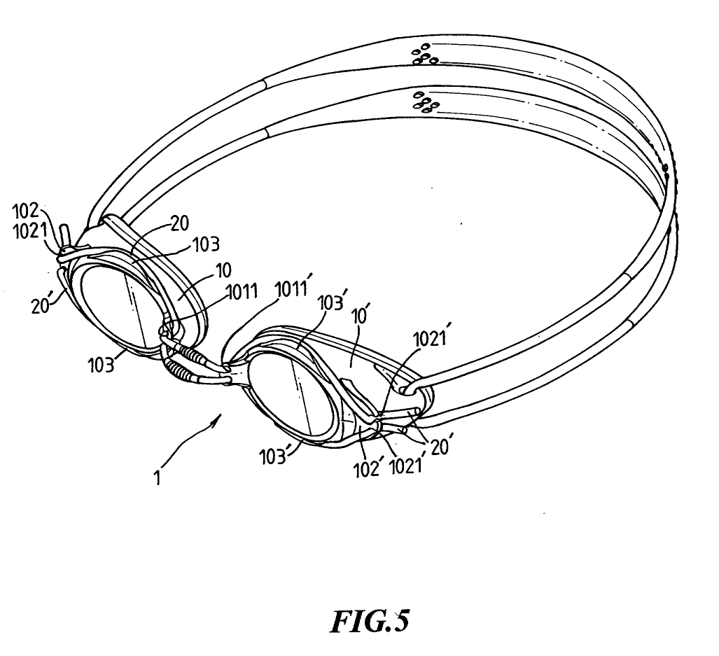

As shown in FIG. 2, the subject invention of swimming goggles 1 comprises the following: two lens frame units 10 and 10′, two strings 20 and 20′, two protective pads 30 and 30′, and a headband device 4. Said two lens frame units 10 and 10′ are positioned left and right and respectively accommodating two lenses 50 and 50′. Said two lens 50 and 50′ are implanted and fixed onto the lens frame units 10 and 10′. On the inside ends of the two lens frame units 10 and 10′ have two first joining units 101 and 101′, while on the outside ends of the two lens frame units 10 and 10′ have two second joining units 102 and 102′. On the first and second joining units 101, 101′ and 102, 102′ wherein the first joining 101 and 101′ are close to the inner of each lens frame 10 and 10′ have a through holes 1011 and 1011′, and the diameter of the through hole 1011 and 1011′ are 2. mm which correspond to a engaging hole which locate are close to the second joining units 102 and 102′ of the outside of lens frame 10 and 10′. The second joining units 102 and 102′ have clasp openings 1021, 1021′ serving to clasp the free ends of the two strings 20 and 20′.respectively. Extending from the upsides and downsides of the lens frame units 10, 10′ correspondingly are clasp pieces 103, 103′. Said clasp pieces 103, 103′ and the lens frame units 10, 10′ are combined to form a fixing groove, as shown in FIG. 5, which serves to firm the winding strings in position. On the lens frame 10, 10′ and next to the second joining units 102, 102′ are two through holes 104, 104′ that serve to accommodate the insertion of the headband device 4.

The two strings 20 and 20′, are made of a flexible material, and the diameter of strings is 1.9 mm which is less than the diameter of the through holes 1011,1011′ which are 2.0 mm, and also less than the diameter of the engaging holes which are 2.0 mm, so both the strings 20 and 20′ could be smoothly pulled through said through holes 1011,1011′ clipped into the clasp piece 102,102′ respectively, and in the meantime, the strings winds round the clasp pieces 103, 103′ on the edges of the two lens frame units 10 and 10′. Appropriate stretches extended from the outside ends of the lens frame units 10, 10′ enable users to convenient handling. Said protective pads 3 are respectively inserted in the far sides of the two lens frames 10 and 10′ away from the lenses 50 and 50′, each has a face contact part 30 or 30′ with flexibility to provide comfortable contact with the user's face and the effects of better suction, and resist the seepage of water.

Please refer to FIGS. 2,3 and 4, the subject invention of swimming goggles is assembled in the following steps: the free end of the two strings 20 and 20′ pass through onto the through holes 1011, 1011′ of the first joining units 101, 101′, then circle the rims of the lens frame units 10, 10′ and inlay onto the clasp pieces 103, 103′. Also, the free ends of the two strings 20 and 20′ are inlay in position onto the second joining units 102, 102′, maintaining an appropriate distance from the two lens frame units 10, 10′, and exposing a small section at the end of the two strings 20 and 20′, so the two lens frame units 10 and 10′ can be joined as one unit.

While adjusting of the span of the invention of swimming goggles, as shown in FIG. 5, just separate one said of each string 20, 20′ from the clasp opening 1021, 1021′ of the second joining unit 102, 102′, and adjust the length of the upper and lower strings 20, 20′ to suit the user's nose bridge contour and provide comfortable contact with the user's face. The span of the nose bridge of the invention of swimming goggles is adjusted by changing the length of the two strings 20 and 20′, thus enabling step-less adjustment to suit the nose bridge configuration of different users who can feel comfortable. Therefore, it can be more convenience and do not effect the span after the adjustment of the span, due to the diameters of two strings 20 and 20′ were less than the through holes 101, 1011′, and the a engaging hole with the clasp opening, so both the strings 20 and 20′ could be smoothly pulled through said through holes 1011, 1011′.

Having proved that the subject invention is capable of achieving the anticipated objective, as described above, it has met the qualification for a patent right. However, the above description has covered merely the preferred embodiment of the subject invention. It is declared herewith that all equivalent modifications and/or variations deriving from the above shall be included in the spirit of the subject claims.

What is claimed is:

1. A type of swimming goggles with step-less adjustment, comprising:
two lens frame units, each accommodate a lens, and at two opposite ends are first joining units and second joining units, wherein the first joining which is located closely to the inner of each lens frame has a through hole, and the second joining units which is located on the outside of each lens frame has a engaging hole with a clasp piece thereon;
two strings which the diameter are less than the through holes and the engaging hole, to pull and position in sequential order along the through holes and a clasp pieces of the first and second joining units of said two lens frame units, maintaining an appropriate span between the two lens frame units;
a headband device, including at least a headband which joined to the second joining unit on the outside of the two lens frame units; thus enabling step-less adjustment to suit the nose bridge configuration of different users who can feel comfortable, and can be more convenient and do not effect the span after adjusting of the span.

2. The swimming goggles with step-less adjustment, as recited in claim 1, wherein an appropriate length of one end of the string is exposed on the outside of the lens frame unit, enabling users to have convenient handling.

3. A type of swimming goggles with step-less adjustment, comprising:
two lens frame units, each accommodating a lens, and at two opposite ends are first joining units and second joining units, wherein the first joining which is located closely to the inner of each lens frame has a through hole, and the second joining units which is located on the outside of each lens frame has a engaging hole with a clasp piece thereon;
at least one string which the diameter was less than the through holes and the a engaging hole, to pull and position in sequential order along the through holes and the clasp pieces of the first and second joining units of said two lens frame units, maintaining an appropriate span between the two lens frame units;
a headband device, including at least a headband which joined to the second joining unit on the outside of the two lens frame units; thus enabling step-less adjustment to suit the nose bridge configuration of different users who can feel comfortable, and can be more convenience and do not effect the span after adjusting of the span.

4. The swimming goggles with step-less adjustment, as recited in claim 3, wherein an appropriate length of one end of the string is exposed on the outside of the lens frame unit, enabling users to have convenient handling.

5. The swimming goggles with step-less adjustment, as recited in claim 4, wherein on the lens frame units and next to the second joining units are headband through holes to facilitate the insertion of the headband.

6. An individual construction of lens frame unit, a component for the composition of swimming goggles, said lens frame unit accommodating a lens frame unit, including: at two opposite ends are first joining units and second joining units, wherein the first joining which is located closely to the inner of each lens frame having a through hole, and the second joining units which is located on the outside of each lens frame has a engaging hole with a clasp piece thereon;
at least one string which the diameter was less than the through holes and the a engaging hole, to pull and position in sequential order along the through holes and the clasp pieces of the first and second joining units of said lens frame units, maintaining an appropriate span between the two lens frame units; and thereby the same two constructions of lens frames, to compose a pair of swimming goggles.
